(19) United States
(12) Patent Application Publication
(10) Pub. No.: US 2015/0272459 A1
(43) Pub. Date: Oct. 1, 2015

(54) SENSOR PROBE ASSEMBLY

(71) Applicant: Neurotopia, Inc., Atascadero, CA (US)

(72) Inventor: Dale Dalke, Atascadero, CA (US)

(21) Appl. No.: 14/739,819

(22) Filed: Jun. 15, 2015

Related U.S. Application Data
(63) Continuation-in-part of application No. 13/551,770, filed on Jul. 18, 2012.

(51) Int. Cl. A61B 5/04

(52) U.S. Cl. CPC A61B 5/04001

(57) ABSTRACT

Preferably, an embodiment of a sensor probe assembly includes at least, a flexible, highly electrically conductive signal sensor component. The flexible, highly electrically conductive signal sensor component provides a main body portion, a plurality of spires protruding in a first direction from a first side surface of the main body portion, and a conductor confinement feature formed on a second side surface of the main body portion. The second side surface is an opposite side surface of the main body portion relative to the first side surface, and in which each of the spires protrude from the main body portion not less than twice a thickness of the main both portion

### RELATED APPLICATION

This application is a continuation-in-part of U.S. patent application Ser. No. 13/551,770 filed on Jul. 18, 2012, entitled “Sensor Probe Assembly.”

### FIELD OF THE INVENTION

The present invention relates to the field of sensors. More particularly, the present invention relates to sensor probe assemblies.

### BACKGROUND OF THE INVENTION

The present invention relates to sensor probe assemblies for use in recording neurophysiological signals. Prior art sensor probe assemblies, have for the most part, depended on the preparation of an area of interest on a cranium of a subject, application of a gel like conductive material, and attachment of the probe to the cranium of the subject at the prepared and gelled site.

As advancements have been made in the field of electronics, it has become desirable to obtain neurophysiological signal data from subjects external to a laboratory or testing facility environment, without the need to prepare and gel a site of interest. Accordingly, improvements in apparatus and methods of providing sensor probes are needed and it is to these needs the present invention is directed.

### SUMMARY OF THE INVENTION

In accordance with preferred embodiments, a sensor probe assembly preferably includes at least a flexible, highly electrically conductive signal sensor component. The flexible, highly electrically conductive signal sensor component provides a main body portion, a plurality of spires protruding in a first direction from a first side surface of the main body portion, and a conductor confinement feature formed on a second side surface of the main body portion. The second side surface is an opposite side surface of the main body portion relative to the first side surface, and in which each of the spires protrude from the main body portion not less than twice a thickness of the main body portion. In a preferred embodiment a sensor tip of the inventive sensor probe assembly is formed from the union of the flexible, highly electrically conductive signal sensor component, and the sensor component support member.

These and various other features and advantages that characterize the claimed invention will be apparent upon reading the following detailed description and upon review of the associated drawings.

### BRIEF DESCRIPTION OF THE DRAWINGS

The present invention is illustrated by way of example and not limitation in the figures of the accompanying drawings, in which like references indicate similar elements and in which:

FIG. 1 is a top plan view of an embodiment exemplary of the inventive sensor probe assembly.

FIG. 2 is a view in elevation of an embodiment exemplary of a conductive pin of the inventive sensor probe assembly of FIG. 1.

FIG. 3 is a front side view in elevation of an embodiment exemplary of the inventive sensor probe assembly of FIG. 1.

FIG. 4 is a front side view in elevation of an embodiment exemplary of the inventive sensor probe assembly illustrative of a flexible, electrically conductive pin securement member and associated plurality of electrically conductive pins matted thereto, of an embodiment exemplary of the inventive sensor probe assembly of FIG. 1.

FIG. 5 is a top plan view of an alternate embodiment exemplary of the inventive sensor probe assembly.

FIG. 6 is a view in front elevation of an alternate embodiment exemplary an electrically conductive pin of the inventive sensor probe assembly of FIG. 5.

FIG. 7 is a front side view in elevation of an alternate embodiment exemplary of the inventive sensor probe assembly of FIG. 5.

FIG. 8 is a front side view in elevation of an alternate embodiment exemplary of the inventive sensor probe assembly illustrative of a flexible, electrically conductive pin securement member and associated plurality of electrically conductive pins matted thereto, of an embodiment exemplary of the inventive sensor probe assembly of FIG. 5.

FIG. 9 is a front elevation view of an embodiment exemplary of an electrically conductive pin of FIG. 6, showing a head portion, a tip portion, and a body portion disposed there between.

FIG. 10 is a front elevation view of an embodiment exemplary of an electrically conductive pin of FIG. 2, showing a head portion having a convex shape, a tip portion, and a body portion disposed there between.

FIG. 11 is a front elevation view of an alternate embodiment exemplary of an electrically conductive pin of FIG. 2, showing a head portion having a concave shape, a tip portion, and a body portion disposed there between.

FIG. 12 is a front elevation view of an embodiment exemplary of an electrically conductive pin of FIG. 2, showing a head portion having a substantially flat top surface, a tip portion, and a both portion disposed there between.

FIG. 13 is a partial cutaway front elevation view of an alternate tip configuration for any of the electrically conductive pins of FIG. 9, 10, 11, or 12.

FIG. 14 is a cross-section, partial cutaway front elevation view of an alternate tip configuration for any of the electrically conductive pins of FIG. 9, 10, 11, or 12.

FIG. 15 is a partial cutaway front elevation view of an alternative tip configuration for any of the electrically conductive pins of FIG. 9, 10, 11, or 12.

FIG. 16 is a partial cutaway front elevation view of an alternate tip configuration for any of the electrically conductive pins of FIG. 9, 10, 11, or 12.

FIG. 17 is a flowchart of a method of producing an embodiment exemplary of the inventive sensor probe assembly of either FIG. 1 or FIG. 5.

FIG. 18 is a bottom plan view of an alternate embodiment of a flexible, highly electrically conductive signal sensor component of an alternate embodiment exemplary of the inventive sensor probe assembly.

FIG. 19 is a top plan view of the alternate embodiment of the flexible, highly electrically conductive signal sensor component of the alternate embodiment exemplary of the inventive sensor probe assembly.

FIG. 20 is a top plan view of an embodiment of a sensor component support member of the alternate embodiment exemplary of the inventive sensor probe assembly.

FIG. 21 is a bottom plan view of an embodiment of the sensor component support member of the alternate embodiment exemplary of the inventive sensor probe assembly.

FIG. 22 is a cross section view in elevation of the embodiment of the flexible, highly electrically conductive signal sensor component of FIG. 18.

FIG. 23 is a cross section view in elevation of the embodiment of the sensor component support member of FIG. 20.

FIG. 24 is a cross section view in elevation of a sensor tip of the inventive sensor probe assembly formed from the union of the flexible, highly electrically conductive signal sensor component of FIG. 18, and the sensor component support member of FIG. 20.

FIG. 25 is a view in elevation of a compressible electrically conductive member communicating with a conductor confinement feature of the flexible, highly electrically conductive signal sensor component of FIG. 18.

FIG. 26 is a view in elevation of the compressible electrically conductive member communicating with an alternate conductor confinement feature of the flexible, highly electrically conductive signal sensor component of FIG. 18.

### DESCRIPTION OF PREFERRED EMBODIMENTS

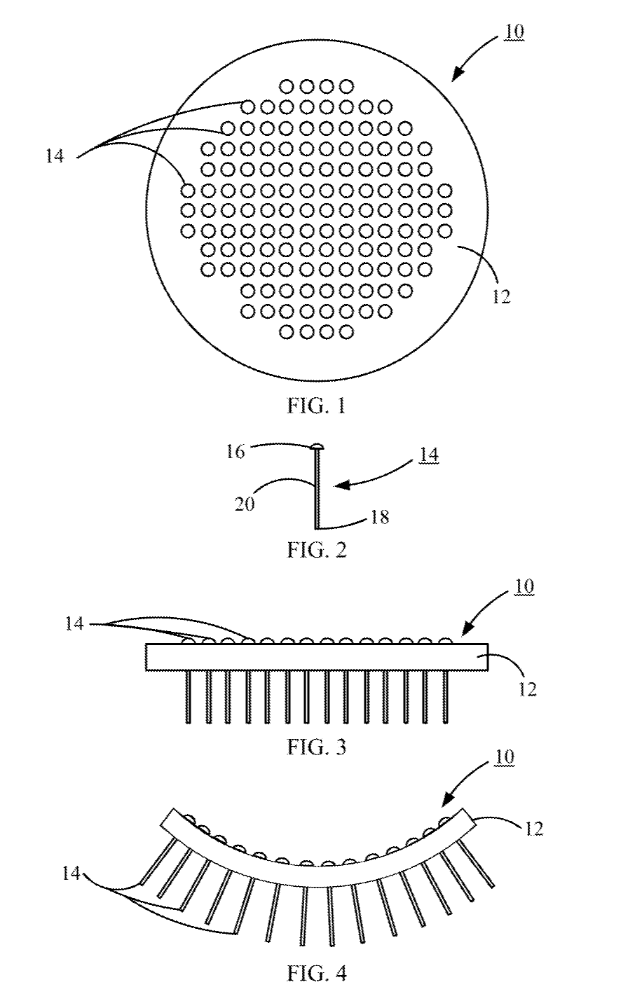

It will be readily understood that elements of the present invention, as generally described and illustrated in the figures herein, could be arranged and designed in a wide variety of different configurations. Referring now in detail to the drawings of the preferred embodiments, a sensor probe assembly 10, of FIG. 1, (also referred to herein as assembly 10) of a first preferred embodiment, while useable for a wide variety of bio-physiological sensing applications, it is particularly adapted for use as neurophysiological signal sensor component. Accordingly, the assembly 10 of the first preferred embodiment, of FIG. 1, will be described in conjunction with the merits of the use of the sensor probe assembly 10 as a neurophysiological signal sensor component.

in a preferred embodiment of FIG. 1, the sensor probe assembly 10 includes at least a conductive pin securement member 12, which hosts a plurality of conductive pins 14. Preferably, the plurality of conductive pins 14 are electrically conductive, and when in pressing contact with the conductive pin securement member 12, as shown by FIG. 3, form the sensor probe assembly 10 that yields a low impedance neurophysiological signal sensor component.

In a preferred embodiment, the conductive pins 14, an example of which is shown by FIG. 2, include at least a head portion 16, as tip portion 18, and as body portion 20 disposed between the head. portion 16 and the tip portion 18. Preferably, each conductive pin 14 is formed from a noncorrosive material, such as stainless steel, titanium, bronze, or a gold plating on a rigid substrate selected from a group including at least polymers and metals. Preferably, the head portion 16 has a diameter greater than the diameter of the body portion 20.

As shown by FIG. 4, the conductive pin securement member 12 is preferably flexible and formed from a polymer. The electrical conductivity of the conductive pin securement member 12 is preferably attained by the inclusion of conductive particles embedded within the polymer. One such combination is a carbon filed silicon sheet material provided by Stockwell Elastomeries, Inc. of Philadelphia, Pa. However, as known in the art, conductive polymers may be formed from a plurality of polymer materials filled with conductive particles, the shape of which may be formed using well known manufacturing techniques that include at least molding, extrusion dies and sliced to thickness, formed in sheets and: die cut; cut with hot wire equipment; high pressure water jets, or steel rule dies.

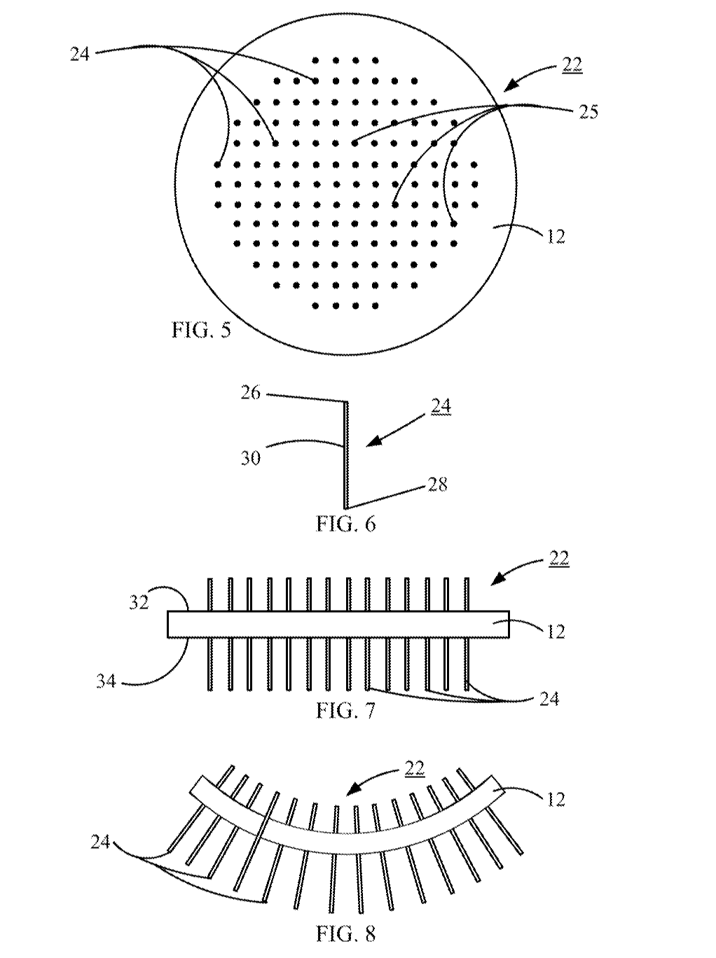

FIG. 5 shows an alternate embodiment of a sensor probe assembly 22, which is preferably formed from the flexible, electrically conductive pin securement member 12, and a plurality of alternate preferred conductive pins 24 cooperating with a corresponding securement pass through aperture 25. The plurality of securement pass through apertures 25 are provided by the flexible, electrically conductive pin securement member 12. As shown by FIG. 6, preferably each alternate preferred conductive pin 24 includes a head portion 26, a tip portion 28, and a body portion 30, wherein the head portion 26 and the tip portion 2$ have diameters substantially equal to the body portion 30. However, a skilled artisan will appreciate that conductive pins may have head, tip and body portion diameters different from one another. For example, the body portion may have a diameter greater than either the tip portion or head portion to accommodate insert molding of the conductive pins into a conductive pin securement member. It is further understood that the conductive pins may take on a profile that includes a bend in the body, tip, or bead portions, as opposed to the cylindrical configuration of any suitable cross section geometric shape of the conductive pins shown by FIG. 2 and FIG. 6. It is still further understood, that the conductive pins may be formed by a plurality of individual components, including without limitation a spring, or may he formed from as coiled or other form of spring alone.

As with the preferred conductive pins 14, the alternate preferred conductive pins 24 are formed from a non-corrosive material, such as stainless steel, titanium, bronze, or a precious metal plating on a rigid substrate selected from a group including at least polymers and metals.

FIG. 7 shows the conductive pins 24 protruding through each the top and bottom surfaces, 32 and 34 respectfully, to accommodate improved conductivity of the alternate sensor probe assembly 22, with mating components. While FIG. 8 shows that the alternate sensor probe assembly 22 preferably retains the flexibility characteristics of sensor probe assembly 10 of FIG. 4.

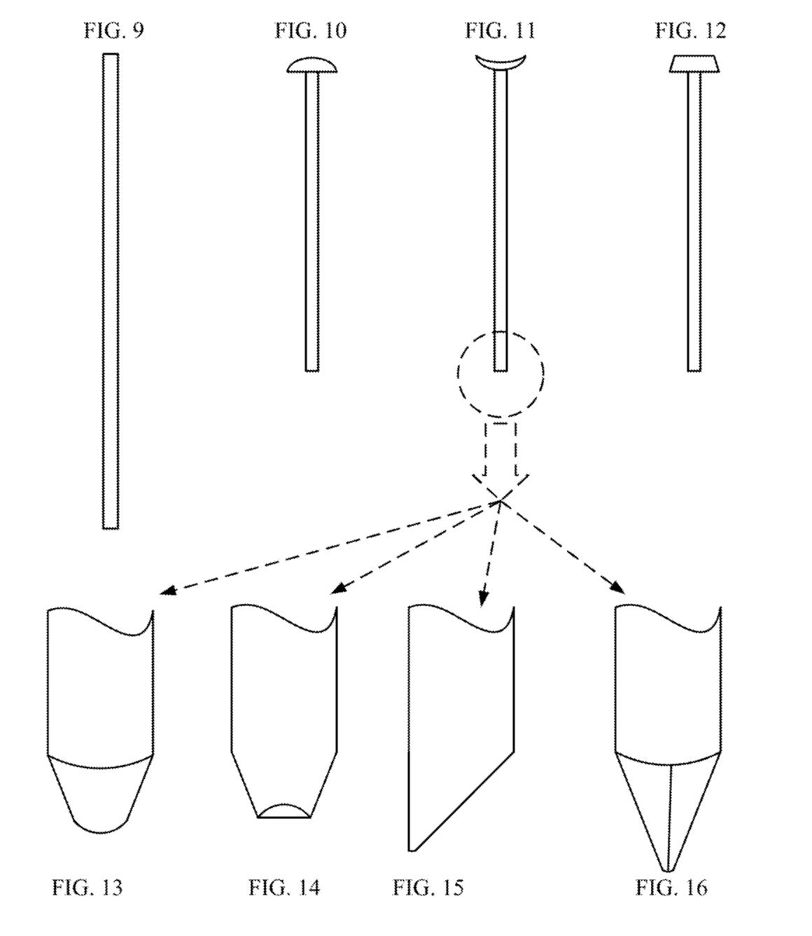

FIGS. 9, 10, 11, and 12 show just a few of a plurality of head configurations suitable for use on conductive pins. The particular configuration selected is a function of the device or component with which the conductive pins electrically cooperate. When a connector is used to interface with the sensor probe assembly, such as 10 or 22, the precise configuration will depend on the type and configuration of the pins associated with the connector, including whether the pins are male or female pins.

FIGS. 13, 14 (a cross section view), 15, and 16 show just a few of a plurality of tip configurations suitable for use on conductive pins. The particular configuration selected is a function of the materials used to form the conductive pins, and the environment in which the conductive pin will be placed. Examples of the use environment include where on the cranium the sensor will be placed, whether hair is present, and the sensitivity of the subject to the tips of the conductive pins.

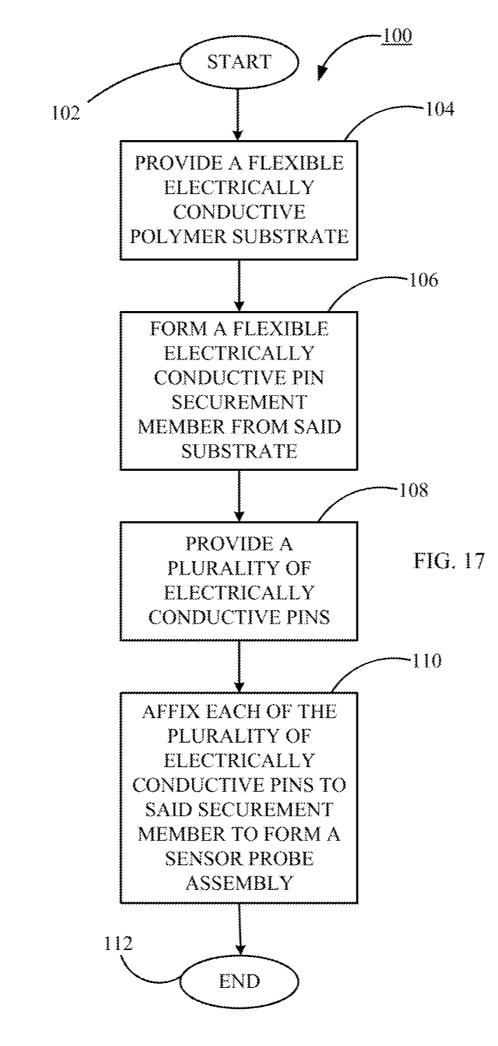

FIG. 17 shows a method 100, of making a sensor probe assembly, such as 10 or 22. The method begins at start step 102, and proceeds to process step 104, where a flexible conductive pin securement material is provided (also referred to herein as a flexible, electrically conductive, polymer substrate). At process step 106, a flexible, electrically conductive, pin securement member (such as 12) is formed from the flexible, electrically conductive, polymer substrate.

The process continues at process step 108, a plurality of electrically conductive pins (such as 14) is provided, At process step 110, each of the plurality of electrically conductive pins are affixed to the flexible, electrically conductive, pin securement member, and the process concludes at end process step 112 with the formation of a sensor probe assembly.

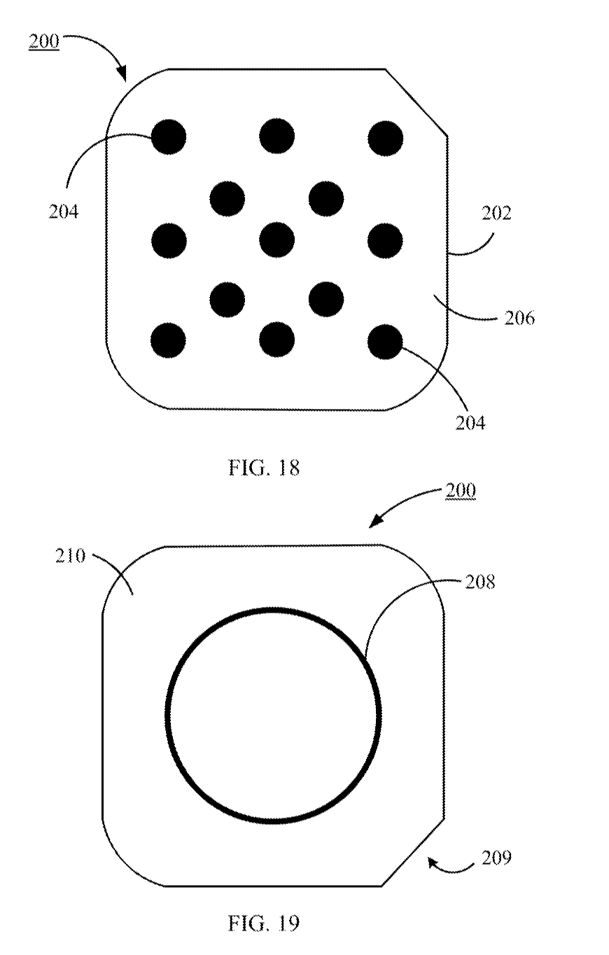

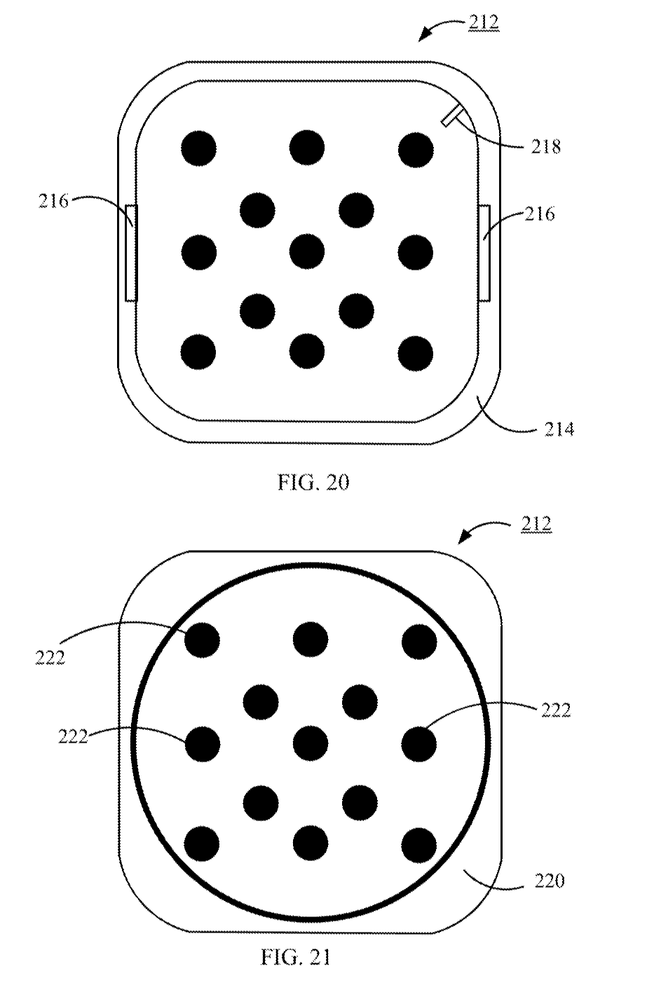

FIG. 18 depicts a bottom plan view of a flexible, highly electrically conductive signal sensor component 200. The flexible, highly electrically conductive signal sensor component 200 preferably provides a main body portion 202, and a plurality of spires 204, protruding in a first direction from a first side surface 206, of the main body portion. While FIG. 19 shows a conductor confinement feature 208, formed on a second side surface 210, of the main body portion 202. The second side surface 210 is an opposite side surface of the main body portion 202, relative to the first side surface 206, and in which each of the spires 204, protrude from the main body portion 202 not less than twice a thickness of the main body portion 202. In a preferred embodiment, as shown by both FIGS. 18 and 19, the flexible, highly electrically conductive signal sensor component 200 preferably provides an orientation feature 209. The orientation feature 209 has been found useful in aligning the flexible, highly electrically conductive signal sensor component 200, with a sensor component support member 212, of FIG. 20.

FIG. 20 depicts a top plan view of the sensor component support member 212. The sensor component support member 212, preferably provides a sensor component confinement feature 214, an attachment feature 216 extending from the sensor component confinement feature 214, an alignment feature 218, extending from the sensor component confinement feature 214. In a preferred embodiment, the alignment feature 216, provides an orientation reference, which in concert with the orientation feature 209, of FIG. 19, promotes a proper alignment of the flexible, highly electrically conductive. signal sensor component 200, with the sensor component support member 212.

FIG. 21 illustrates a bottom surface 220, of the sensor component support member 212, and a plurality of spire apertures 222, provided by the sensor component support member 212. In a preferred embodiment, each spire aperture of the plurality of spire apertures 222 cooperates with its corresponding spire 204, of the plurality of spires 204, of FIG. 18, to form a sensor tip 224, as shown by FIG. 24.

FIG. 22 shows a cross section view in elevation of the embodiment of the flexible, highly electrically conductive signal sensor component 200, which in a preferred embodiment, provides the main body portion 202; the plurality of spires 204, protruding in a first direction from the first side surface 206, of the main body portion 202; and the conductor confinement feature 208, formed on a second side surface 210, of the main body portion 202. It is noted that preferably, that a length 226 of each spire 204 is greater than twice a thickness 228, of the main body portion 206, of the flexible, highly electrically conductive signal sensor component 200, and that each spire of the plurality of spires 204 are of a common length.

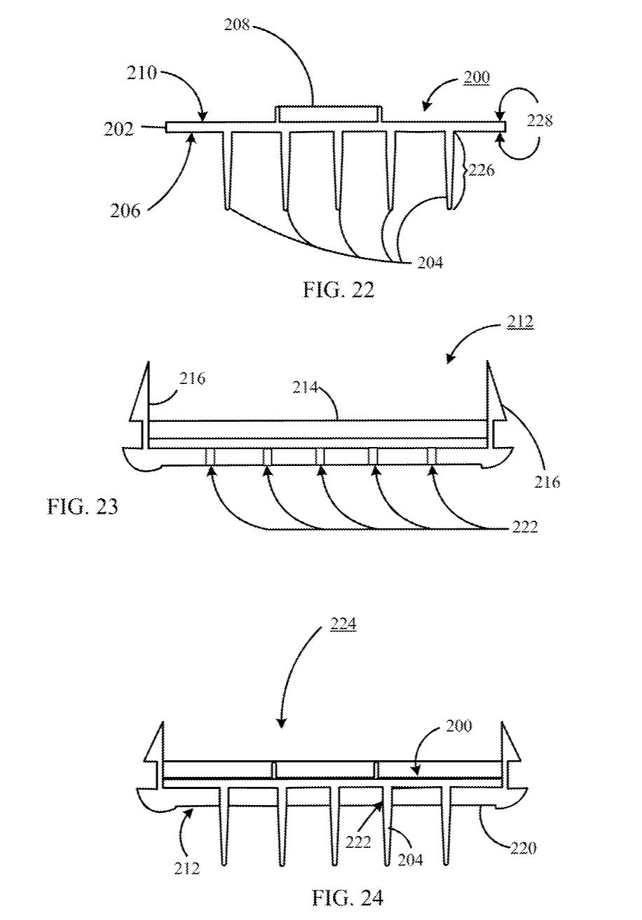

FIG. 23 shows a cross section view in elevation of the preferred embodiment of the sensor component support member 212, of FIG. 20, to provide a further understanding of: the sensor component confinement feature 214; the attachment feature 216 that protrudes from the sensor component confinement feature; and the plurality of spire apertures 222.

The joinder of the flexible, highly electrically conductive signal sensor component 200, with the sensor component support member 212, forms the preferred sensor tip 224, as shown by FIG. 24. As further shown by FIG. 24, preferably each spire 204, protrudes through its corresponding spire aperture 224, such that greater than one half the length 226, as seen in FIG. 22, of each spire 204 extends beyond the bottom surface 220, of the sensor component support member 212.

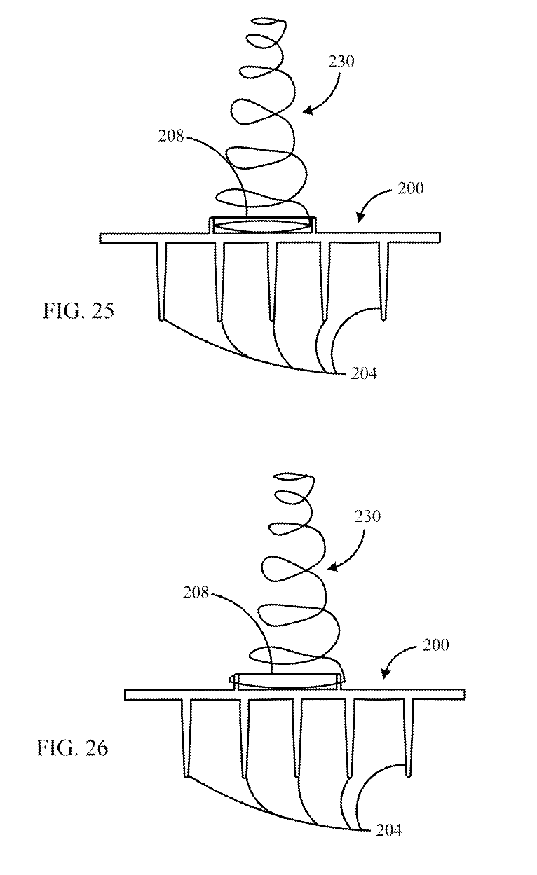

FIGS. 25 and 26 each show compressible electrically conductive member 230, communicating with the conductor confinement feature 208, of the flexible, highly electrically conductive signal sensor component 200. FIG. 25 shows the compressible electrically conductive member 230, is supported by the internal circumference of the conductor confinement feature 208. While FIG. 26, shows the compressible electrically conductive member 230, is supported by the external circumference of the conductor confinement feature 208.

As will be apparent to those skilled in the art, a number of modifications could be made to the preferred embodiments which would not depart from the spirit or the scope of the present invention. While the presently preferred embodiments have been described for purposes of this disclosure, numerous changes and modifications will be apparent to those skilled in the art. Insofar as these changes and modifications are within the purview of the appended claims, they are to be considered as part of the present invention.

What is claimed is:

1. a sensor probe assembly comprising, a flexible, highly electrically conductive signal sensor component, the flexible, highly electrically conductive signal sensor component provides a main body portion, a plurality of spires protruding in a first direction from a first side surface of the main body portion, and a conductor confinement feature formed on a second side surface of the main body portion, wherein the second side surface is an opposite side surface of the main body portion relative to the first side surface, and in which each of the spires protrude from the main body portion not less than twice a thickness of the main body portion.

2. The sensor probe assembly of claim 1, in which the flexible, highly electrically conductive signal sensor component further providing an orientation feature.

3. The sensor probe assembly of claim 2, in which the main body portion, the plurality of spires, and conductor confinement feature are formed from a common material.

4. The sensor probe assembly of claim 3, in which the main body portion, the plurality of spires, and conductor confinement feature are formed as a single, unified highly electrically conductive signal sensor component from the common material.

5. The sensor probe assembly of claim 4, in which the common material, the main body portion, the plurality of spires, and conductor confinement feature formed as the single, unified highly electrically conductive signal sensor component is a conductive polymer.

6. The sensor probe assembly of claim 5, in which the conductive polymer is carbon particle impregnated polymer.

7. The sensor probe assembly of claim 6, in which the polymer is silicone.

8. The sensor probe assembly of claim 1, further including at least a sensor component support member communicating with the flexible, highly electrically conductive signal sensor component.

9. The sensor probe assembly of claim 8, in which the sensor component support member provides at least a sensor component confinement feature, the sensor component confinement feature cooperates with and supports a perimeter of the flexible, highly electrically conductive signal sensor component.

10. The sensor probe assembly of claim 9, in which the sensor component support member further provides at least an attachment feature protruding from the sensor component, confinement feature and adjacent the perimeter of the flexible, highly electrically conductive signal sensor component.

11. The sensor probe assembly of claim 10, in which the sensor component support member still further provides a plurality of spire apertures, each spire aperture communicating with a corresponding spire of the plurality of spires, and in which each spire protrudes through its corresponding spire aperture such that greater than one halt a length of each spire extends beyond a bottom surface of the sensor component support member.

12. The sensor probe assembly of claim 11, in which each spire of the plurality of spires is of a common length.

13. The sensor probe assembly of claim 12, in which the sensor component support member is formed from a non-conductive polymer.

14. The sensor probe assembly of claim 13, in which the sensor component support member provides an alignment feature extending from the sensor component confinement feature and cooperating with an orientation feature of the flexible, highly electrically conductive signal sensor component.

15. The sensor probe assembly of claim 7, further including at least a sensor component support member communicating with the flexible, highly electrically conductive signal sensor component.

16. The sensor probe assembly of claim 15, in which the sensor component support member provides at least a sensor component confinement feature, the sensor component confinement feature cooperates with and supports a perimeter of the flexible, highly electrically conductive signal sensor component.

17. The sensor probe assembly of claim 16, in which the sensor component support member further provides at least an attachment feature protruding from the sensor component confinement feature and adjacent the perimeter of the flexible, highly electrically conductive signal sensor component.

18. The sensor probe assembly of claim 17, in which the sensor component support member still further provides a plurality of spire apertures, each spire aperture communicating with a corresponding spire of the plurality of spires, and in which each spire protrudes through its corresponding spire aperture such that greater than one half a length of each spire extends beyond a bottom surface of the sensor component support member.

19. The sensor probe assembly of claim 18, in which each spire of the plurality of spires is of a common length.

20. The sensor probe assembly of claim 19, in which the sensor component support member is formed from a non-conductive polymer, and in which the sensor component support member provides an alignment feature extending from the sensor component confinement feature and cooperating with the orientation feature of the flexible, highly electrically conductive signal sensor component.
